(19) United States
(12) Patent Application Publication
(10) Pub. No.: US 2003/0004533 A1
(43) Pub. Date: Jan. 2, 2003

(54) Bioactive polymer vaso-occlusive device

(75) Inventors: Martin S. Dieck, Cupertino, CA (US); Christopher G.M. Ken, San Mateo, CA (US); Tina J. Patel, San Carlos, CA (US)

Correspondence Address: BANNER & WITCOFF, 1001 G STREET N W, SUITE 1100, WASHINGTON, DC, 20001, US

(73) Assignee: Concentric Medical, Mountain View, CA

(21) Appl. No.: 10/138,529

(22) Filed: May 6, 2002

Related U.S. Application Data
(60) Provisional application No. 60/288,459, filed on May 4, 2001.

(51) Int. Cl. A61M 29/00

(52) U.S. Cl. 606/191

(57) ABSTRACT

Methods and apparatus for performing vaso-occlusion. The apparatus includes a vaso-occlusion device made of a non-metal material. The non-metal can be one or more polymers, and one or more of the polymers can biodegrade. The device can also comprise a bioactive agent and a radio pacifier. The device can be delivered as a solid or as a liquid injectable material. The methods provide a treatment for abnormal blood flow by implanting such a device at the site of abnormal blood flow. Additionally, is provided methods of making the vaso-occlusive devices.

### CROSS-REFERENCE TO RELATED APPLICATION

This application claims benefit under 37 CFR §1.78 of provisional application No. 60/288,459, filed May 4, 2001. The full disclosure of the application is incorporated herein by reference.

### FIELD OF THE INVENTION

The present invention relates to vaso-occlusive devices and methods of treating conditions manifesting abnormal blood flow employing the vaso-occlusive devices.

### BACKGROUND OF THE INVENTION

Ruptured blood vessels in the brain cause an acute condition known as hemorrhagic stroke. Ruptures or strokes can occur with a number of vascular abnormalities including arterio venous malformation (AVM), fistula, aneurysm (a ballooning of the arterial wall), or a burst blood vessel. In addition, abnormal vasculature is generated in the process of tumor growth and tumors including brain tumors are highly vascularized entities requiring larger than normal blood flow to sustain the tumor.

Endovascular therapy for vaso-occlusion has included injectable agents, balloon-type occlusive devices, and mechanical vaso-occlusive devices such as metal coils. A description of these agents and devices is included in the background section of U.S. Pat. No. 4,994,069.

Currently, coils for aneurysms and polyvinyl alcohol (PVA) particles for AVMs are FDA approved preventative therapies. Cyanoacrylate glue for AVMs is also proposed and pending approval.

Over 400,000 persons worldwide, and 125,000 persons in the U.S. annually experience some form of hemorrhagic stroke or blood vessel rupture in the brain. A need exists in the medical community and the field of interventional neurology for devices and/or agents that can be effectively used in interventional radiology treatments at sites of abnormal blood flow.

Current embolic devices are made from platinum, tungsten, and stainless steel. All these materials create very minimal biological response when place in the body. The disadvantage of using metals which are inert in the body are that they operate as purely mechanical emboli and create a thrombotic response by providing stagnation and turbulence in the area of abnormal blood flow. Another disadvantage of using metal embolic devices is that the metal in the device can create artifacts under magnetic resonance imaging. A need exists for devices that can provide a biological response in addition to mechanical blockage would be of great use to the medical community in this regard.

### SUMMARY OF THE INVENTION

An object of the invention is to provide a non-metal vaso-occlusive device. Accordingly is provided, a non-metal vaso-occlusive device for implantation into the vasculature of a patient to occlude abnormal blood flow comprising:

a material comprising a polymer or combination of polymers in a solid form, wherein the material is configured in a pre-implantation shape before implantation and assumes a vaso-occluding shape after implantation.

The polymer or polymers can be selected from the group consisting of polyacrylamide (PAAM), poly(N-isopropylacrylamine) (PNIPAM), poly(vinylmethylether), poly(ethylene oxide), poly(vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic Lactic Acid (PGLA), a copolymer, and a blend of two or more polymers.

The solid polymer can be a natural polymer.

The natural polymer can be selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, pectin, elastin, keratin, a copolymer, and a blend of polymers.

The pre-implantation shape can comprise a shape selected from the group consisting of a strip, a rod, a sheet, a roll, a tube, a ribbon, a string, and a coil.

The vaso-occluding shape can comprise a shape selected from the group consisting of a coil, a coiled coil, a circle, a half circle, a cone, a twisted sheet, a rod of random bends, and a helix.

The vaso-occluding device can further comprise a bioactive agent integrated into or coating the solid material.

The bioactive agent can comprise a bioactive agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The bioactive agent can comprise a tissue adhesion factor and the tissue adhesion factor is selected from the group consisting of fibrin, collagen, albumin, cyanoacrylate, fibrinogen, chitosan, and gelatin-genipin.

The vaso-occlusive device can further comprise a radio pacifier.

The radio pacifier comprises an agent that provides visibility of the device under X-ray or other imaging technology.

The radio pacifier can comprise a contrast media or a metal powder.

One or more of the polymers of the device comprising the solid material can comprise a biodegradable polymer.

The invention also includes a vaso-occlusive device for implantation into the vasculature of a patient to occlude abnormal blood flow comprising:

a liquid injectable polymer or combination of polymers for delivery to a site of abnormal blood flow upon which delivery the polymer polymerizes or precipitates to assume a vaso-occluding solid shape that occludes abnormal blood flow.

The polymer or polymers are selected from the group consisting of polyacrylamide (PAAM), poly(N-isopropylacrylamine) (PNIPAM), poly(vinylmethylether), poly(ethylene oxide), poly(vinylalcohol), poly(ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic Lactic Acid (PGLA), a copolymer, and a blend of two or more polymers.

The injectable polymer can be a natural polymer.

The natural polymer can be selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, pectin, elastin, keratin, a copolymer, and a blend of polymers.

The vaso-occlusive device can further comprise a bioactive agent integrated into the injectable polymer.

The bioactive agent can comprise a bioactive agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The bioactive agent can comprise a tissue adhesion factor and the tissue adhesion factor is selected from the group consisting of fibrin, collagen, albumin, cyanoacrylate, fibrinogen, chitosan, and gelatin-genipin.

One or more polymers comprising the resulting solid polymer can be biodegradable.

The invention also includes a method of treating a patient having abnormal blood flow comprising:

implanting into the vasculature of the patient at the site of abnormal blood flow a material comprising a polymer or combination of polymers, wherein the material is either a liquid injectable that polymerizes to a solid or precipitates as a solid upon placement in the patient or is a solid material configured in a pre-implantation shape before implantation and changes to a vaso-occluding shape after implantation.

The material can comprise a polymer or combination of polymers selected from the group consisting of polyacrylamide (PAAM), poly(N-isopropylacrylamine) (PNIPAM), poly(vinylmethylether), poly(ethylene oxide), poly(vinylalcohol), poly(ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA)), Polyacrylonitrile (PAN), Polyvinylacrylate (PVA), Polyvinylpyrrolidone (PVP), Polyglycolic Lactic Acid (PGLA), a copolymer, and a blend of two or more polymers.

The material can be a natural polymer. The natural polymer can be selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, pectin, elastin, keratin, a copolymer, and a blend of polymers.

The material implanted in the patient can comprise a bioactive agent.

The bioactive agent can comprise a bioactive agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, and an anti-cancer factor. protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The bioactive agent can comprise a tissue adhesion factor and the tissue adhesion factor is selected from the group consisting of fibrin, collagen, albumin, cyanoacrylate, fibrinogen, chitosan, and gelatin-genipin.

The invention also provides a method of making a vaso-occlusive device for occluding abnormal blood flow comprised of a non-metal solid material comprising:

configuring the non-metal material into a pre-implantation shape, wherein upon implantation into a patient at a site of abnormal blood flow the material assumes a vaso-occluding shape.

The non-metal material can be selected from the group consisting of polyacrylamide (PAAM), poly(N-isopropylacrylamine) (PNIPAM), poly(vinylmethylether), poly(ethylene oxide), poly(vinylalcohol), poly(ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic Lactic Acid (PGLA), a copolymer, and a blend of two or more polymers.

The non-metal material can be a natural polymer. The natural polymer can be selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, pectin, elastin, keratin, a copolymer, and a blend of polymers.

The method of making the device can further comprise integrating into or coating the nonmetal material with a bioactive agent.

Coating or integrating can comprise a process selected from the group consisting of ion implantation, vapor deposition, plasma deposition, coating, jacketing, weaving, braiding, spraying, dipping, and spinning.

The bioactive agent can comprise a bioactive agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The bioactive agent can comprises a tissue adhesion factor selected from the group consisting of fibrin, collagen, albumin, cyanoacrylate, fibrinogen, chitosan, and gelatin-genipin.

One or more of the polymers can be biodegradable.

The method can further comprise mixing a radio pacifier into the material or coating the vaso-occlusive device with a radio pacifier.

The invention further provides a method of making a vaso-occlusive device for occluding abnormal blood flow in a patient comprising:

providing a liquid injectable polymer material that polymerizes to a solid or precipitates to a solid upon placement in the patient.

The liquid injectable polymer material can be selected from the group consisting of polyacrylamide (PAAM), poly(N-isopropylacrylamine) (PNIPAM), poly(vinylmethylether), poly(ethylene oxide), poly(vinylalcohol), poly(ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic Lactic Acid (PGLA), a copolymer, and a blend of two or more polymers.

The material can be a natural polymer. The natural polymer can be selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, pectin, elastin, keratin, a copolymer, and a blend of polymers.

The method can further comprise integrating into the liquid injectable material a bioactive agent. The bioactive agent can comprise a bioactive agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anticancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The bioactive agent can comprise a tissue adhesion factor and the tissue adhesion factor is selected from the group consisting of fibrin, collagen, albumin, cyanoacrylate, fibrinogen, chitosan, and gelatin-genipin.

One or more polymers can be biodegradable. The device used in the method can further comprise a radio pacifier mixed into or coating the vaso-occlusive device.

### BRIEF DESCRIPTION OF THE DRAWINGS

FIG. 1A shows a spherical coil; FIG. 1B shows a vaso-occluding coiled coil shape.

### DETAILED DESCRIPTION OF THE DRAWINGS

The following embodiments and examples are offered by way of illustration and not by way of limitation.

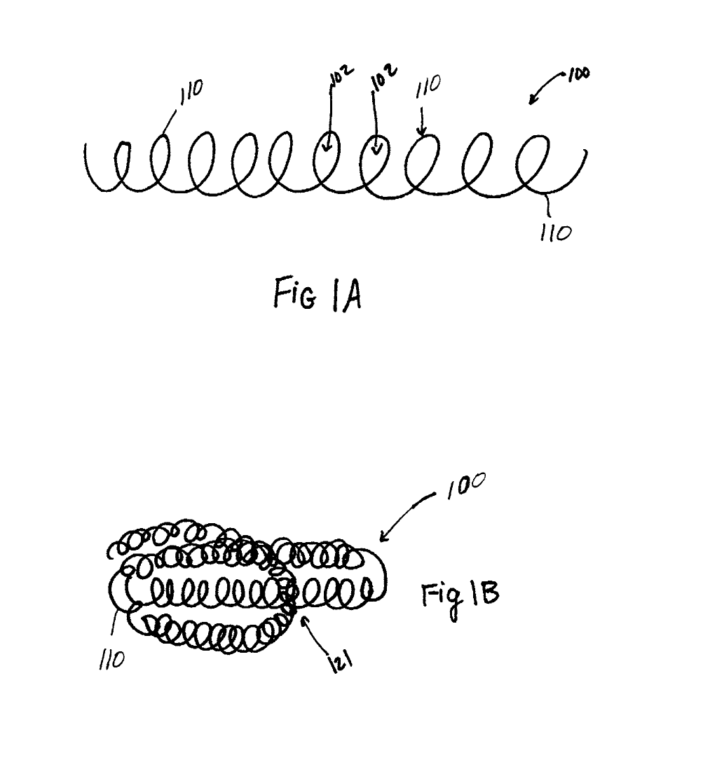

Turning to the Figures, FIG. 1A shows a vaso-occlusive device 100. As depicted, the device 100 includes a generally spherical coil 110. The spherical coil 110 can have a pre-implantation shape as shown in FIG. 1A. In one embodiment, the coil 110 can be helical or extend in a straight line (linear). The spherical coil 110 can also have a vaso-occluding shape as shown in FIG. 1B. The vaso-occlusive shape can include a conventional coil shape or a tangled coil shape (FIG. 1B). Upon implantation, the spherical coil 110 changes from its pre-implantation shape to its vaso-occluding shape.

The coiled or tangled coil 110 winds back on itself crossing itself 121, possibly interlocking and generally complicating the form of the device 100. The joints of the spherical coil 110 can contain further complicating members, such as, for example, extending fibers or fringe. An internal tube 102 created by the coil 110 can be further filled either with another smaller coil or a malleable rod with notches or contours or the like. The spacing and winding (tight or loose or in-between) can vary and is not critical, but depends rather on the polymer selected to form the device.

While a spherical coil shape is depicted in the figures, there are actually many possible, likely, useful, and considered shapes for both the pre-implantation and subsequently formed vaso-occluding shape of the vaso-occlusive device 100. Examples of permissible shapes include those shapes such as, for example, a knotted and tangling coil as described in Ritchart U.S. Pat. No. 4,994,069; a helical coil in a sinusoidal wave configuration, Chee U.S. Pat. No. 5,304,194; a vaso-occlusion braid of woven fibers, Engleson U.S. Pat. No. 5,423,849; a vaso-occlusive coil which is segmented onto which a fibrous woven or braided tubular covering or element is attached, Phelps U.S. Pat. No. 5,522,822; thrombogenic fibers in a central region containing a majority of these fibers upon ejection from the catheter, Mirigian U.S. Pat. No. 5,549,624; helically wound coil which helix is wound in such a way as to have multiple axially offset longitudinal or focal axes, Mariant U.S. Pat. No. 5,639,277; helical metallic coil having a plurality of axially spaced windings and a plurality of strands of a thrombogenic polymer extending axially through the central core of the coil, Snyder U.S. Pat. No. 5,658,308; proximal portion sufficiently flexible to fold on itself, Kupiecki U.S. Pat. No. 5,669,931; a vaso-occlusive helical metal coil having a thermoplastic polymer plug at one end or both, Gia U.S. Pat. No. 5,690,667; complex helically wound coil made up of pre-implantation helically wound coil which is wound in a vaso-occluding shape which is itself a series of helical turns, Wallace U.S. Pat. No. 5,733,329; a variable stiffness coil, Samson U.S. Pat. No. 5,766,160; a conical tipped cylindrical device with filamentary material, Wallace U.S. Pat. No. 5,957,948; helix in a tangled mass, Kupiecki U.S. Pat. No. 6,168,592; the shapes described in Berenstein et al, U.S. Pat. No. 5,826,587; the 3-dimensional in-filling vaso-occlusive coil of Mariant U.S. Pat. No. 5,957,948; the coil depicted in Engleson U.S. Pat. No. 6,024,754, and the multilayered vaso-occlusive coils of Ken et al, U.S. Pat. No. 6,033,423.

The shape of the vaso-occlusive device 100 can take into account the pattern by which the material will degrade, if it degrades, e.g. especially where the device 100 is constructed of one or more materials that biodegrade at different rates, and especially if two or more polymers in the device degrade at different rates. In general the pre-implantation shapes of the coil 110 can be but are not limited to a strip, rod, sheet, roll, tube, ribbon, string or a coil. As mentioned above, the vaso-occluding shapes of the coil 110 can be but are not limited to a coil, a coiled coil, a circle, a half circle, a cone, a twisted sheet, a rod of random bends, or a helix. A non-degrading device 100 can provide a matrix or structure for vaso-occlusion in the patient.

The material used in the vaso-occlusive device 100 of the shape illustrated in FIG. 1A or 1B or any of the shapes just listed should be biocompatible and can be any solid non-metal material. In an embodiment, the coil 120 is formed of a biodegradable material. In an alternative embodiment, less than the entire coil 120 is formed of the biodegradable material. In either embodiment, for example, the non-metal material can comprise a polymer or combination of polymers in a solid form (e.g. a single polymer or a combination of two or more polymers either as a copolymer or as a blend). Suitable definitions for the terms biocompatible and biodegradable are found in Katz, Medical Devices and Diagnostic Industry, January 2001, “Developments in Medical Polymers for Biomaterials Applications”, pp 122-132. Materials for use in making the vaso-occlusive devices are also describe in Katz.

The vaso-occlusive device 100 for implantation into the vasculature of a patient to occlude abnormal blood flow comprising a solid material comprises a polymer or combination of polymers in a solid form. As discussed above, the solid material is configured in a pre-implantation shape before implantation (including shapes described above) and can change into a vaso-occluding shape after implantation (also as described above. The pre-delivery material can also be a liquid injectable that becomes a solid after injection into the patient, either by polymerizing to a solid or precipitating to a solid. The resulting solid in either case assumes some kind of vaso-occluding shape, including but not limited to amorphous shapes. Polymers for the vaso-occlusive device can be, e.g. polyacrylamide (PAAM), poly(N-isopropylacrylamine) (PNIPAM), poly(vinylmethylether), poly(ethylene oxide), poly(vinylalcohol), poly(ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), or Polyvinylpyrrolidone (PVP), Polyglycolic Lactic Acid (PGLA) or copolymers of these polymers, or blends of these polymers. The PGLA disclosed herein is formed by mixing PGA and PLA in ratios of 99.9:00.1 to 50:50.

Natural polymers can be used to make up the device 100, including such natural polymers as, e.g. collagen, silk, fibrin, gelatin, hyaluron, cellulose, chitin, dextran, casein, albumin, ovalbumin, heparin sulfate, starch, agar, heparin, alginate, fibronectin, fibrin, pectin, elastin, keratin, copolymers of these natural polymers, or blends of these natural polymers.

The vaso-occlusive device 100 can also comprise a bioactive agent that is reactive at the site of implantation. For example, the bioactive agent may promote maintaining the device at the site of abnormal blood flow, may promote regrowth of a damaged vascular wall, may help to heal the site, may inhibit continued or re-vascularization, may inhibit or regress tumor growth, and such like biological activities at the site of implantation or abnormal blood flow.

The bioactive agent can be any bioactive agent capable of reacting at the site of implantation of the vaso-occlusive device. Thus, the bioactive agent can be, for example, but not limited to, a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anticancer factor, a monoclonal antibody, a monoclonal antibody against a growth factor, a drug, a drug producing cell, a cell regeneration factor, a progenitor cell of the same type as vascular tissue, or an a progenitor cell that is histiologically different from vascular tissue. The bioactive agent can be a mixture of active agents, e.g. a drug and an antibody, or any effective combination of one or more bioactive agents that can work together or independently at the site of implantation to effect positive biological activity. The bioactive agent may be delivered in a microsphere encapsulating e.g. a viral vector having a gene for expression at the site of implantation.

The bioactive agent can comprise a tissue adhesion factor and the tissue adhesion factor is selected from the group consisting of fibrin, collagen, albumin, cyanoacrylate, fibrinogen, chitosan, and gelatin-genipin.

Synthesis or formulation of a bioactive agent selected can be facilitated generally as is practiced with the agent in laboratory or medicinal contexts, e.g. as demonstrated in standard or published protocols and assays.

The vaso-occlusive device 100 can also comprise a radio pacifier. The radio pacifier can comprise an agent that provides visibility of the device under X-ray or other imaging technology such as CT scans, MRIs and flouroscopy. The radio pacifier permits the device 100 to be monitored and detected once inside the patient. The radio pacifier can comprise, for example, a contrast media or a metal powder, but is not limited to these items. The metal powder can be, for example, titanium, tungsten, gold, bismuth, barium sulfate or tantalum powder. Additionally, the radio pacifier includes a gadolinium-based MRI contrast agent. These agents can include, but are not limited to, Gadopentetate, Gadopentetate dimeglumine (Gd DTPA or Magnevist (R)), Gadoteridol (Gd HP-DO3A or ProHance (R)), Gadodiamide (Gd DTPA-BMA or Omniscan (R)), Gadoversetamide (Gd DTPA-BMEA or OptiMARK (R)), Gd-DOTA (Magnevist (R) or Dotarem (R)), Gd-DTPA labeled albumin, and Gd-DTPA labeled dextran.

In an embodiment, the coil 110 is delivered to the surgeon, other practitioner or attendant in pre-cut or pre-formed lengths. In this embodiment, each coil is cut to a predetermined length. For example, the length of the coil 110 of the vaso-occlusive device 100 as it is delivered can be in the range from about 1 mm to about 5 meters. In a preferred embodiment, the pre-cut lengths of the coils 110 of the vaso-occlusive device 100 for delivery to the patient can be in a range from about 1 mm to about 10 mm. In an embodiment, the dimensions of the device 100 can be from about 0.125 mm to about 12.50 mm, or the outside diameter of objects suitable for passing through a delivery device to a site of abnormal bleeding. The diameter of the vaso-occlusive device 100 once it is delivered and after it has assumed its vaso-occluding shape (FIG. 1B) can be in a range from about 1 mm to about 50 mm.

The vaso-occlusive device 100 having pre-implantation shape and then a vaso-occluding shape can be delivered to the site of abnormal blood flow e.g. by a catheter or pushing device having a lumen for delivering the vaso-occlusive device. The vaso-occlusive device can also be delivered e.g. as described in U.S. Pat. Nos. 4,994,069; USPN 5,304,194; USPN 5,423,849; USPN 5,522,822; USPN 5,549,624; Mariant USPN 5,639,277; USPN 5,658,308; USPN 5,669,931; USPN 5,690,667; USPN 5,733,329; USPN 5,766,160; USPN 5,957,948; USPN 6,168,592; USPN 5,826,587; USPN 5,957,948; USPN 6,024,754, or USPN 6,033,423.

As discussed above, in an embodiment, the vaso-occlusive device 100 is deliverable as a liquid injectable material to the site of abnormal bleeding where the liquid polymerizes to a solid or precipitates into a vaso-occluding shape as a solid to occlude the abnormal blood flow. The liquid injectable material can include one or more of the natural or non-natural polymer discussed above. The liquid injectable material may also comprise one or more of the bioactive agent also discussed above. As the biodegradable polymer degrades, the bioactive agent is released at the site of implantation to promote whatever bioactivity the agent is capable of. The liquid injectable can also comprises a radio pacifier as described earlier. One or more polymers comprising the liquid injectable material can also be biodegradable after implantation in the body.

U.S. Pat. No. 5,808,012 describes a process usable with the present invention by which proteins and other bioactive agents can be incorporated into a polymer during a forming process such as extrusion, molding or casting.

U.S. Pat. No. 6,184,348 describes production of novel polymers using recombinant techniques, and also integration of bioactive agents potentially useful at a site of implantation in the patient. This production can be used with the present invention.

The present invention also comprises a method of treating a patient having abnormal blood flow at a particular site in the body. The device 100 has a pre-implantation shape that changes to a post-implantation vaso-occluding shape can be delivered as described above. Alternatively, the device formed of a liquid injectable material can be delivered as described above. The liquid injectable material as described above can be injected into the site of abnormal blood flow so that it will polymerize or precipitate there, and promote subsequent blood flow occlusion. The method can further include that the material implanted comprises a bioactive agent, such as, for example, those listed herein. The method can also further comprise that the injected or implanted non-metal polymeric material biodegrades in the patient. Thus, one or more polymers that make up the solid material can biodegrade in the patient. Material that does not biodegrade can remain in the patient as a matrix or framework for blood flow occlusion and other biological responses such as healing and rebuilding normal vasculature.

The vaso-occlusive device 100 used in the method is designed for implantation into the vasculature of a patient. The implantation site can be any site of abnormal blood flow in the patient. The abnormal blood flow can be caused by an aneurysm, a ruptured blood vessel, an arterio venous malformation (AVM), fistula, or a benign or malignant tumor. Tumors are in part characterized by a highly vascularized state. Otherwise untreatable tumors are particularly contemplated for treatment by implantation of the vaso-occlusive device of the invention. Use of a radio pacifier in the device provides the opportunity to image and locate the device at a later date.

Additionally, the invention embodies a method of making the vaso-occlusive device 100 as described above. That method includes configuring a non-metal material (which, as discussed above, can be one polymer or a blend, or copolymer of two or more polymers) into a pre-implantation shape. The pre-implantation shape configures into a vaso-occluding, generally more complicated shape, e.g. a coil becomes a coiled coil or a tangled coil, etc. An example of forming a pre-implantation structure using a polymer is described in Pathak, et al, U.S. Pat. No. 6,176,871. The materials used in the making of the device 100 can be the same natural and non-natural polymers listed above, and the like, and also the material is not limited to these selections. One or more of the polymers comprising the material can be biodegradable. Bioactive agents listed herein and the like (and not limited to these) can be integrated into the biodegradable material, for release after the device 100 is implanted, and possibly during biodegradation of the device. Methods of making the device 100 comprising a liquid injectable material will comprise formulating the liquid injectable polymer or plastic and optionally incorporating into the liquid composition a bioactive agent. The mixture will be injected into the patient where it polymerizes to a solid or precipitates to a solid, occludes abnormal blood flow, and optionally subsequently degrades. The material can also further comprises a radio pacifier, as described herein.

All publications, patents and patent applications cited in this specification are herein incorporated by reference as if each individual publication, patent or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A non-metal vaso-occlusive device for implantation into the vasculature of a patient to occlude abnormal blood flow comprising:
a material comprising a polymer or combination of polymers in a solid form, wherein the material is configured in a pre-implantation shape before implantation and assumes a vaso-occluding shape after implantation.

2. A vaso-occlusive device as in claim 1, wherein the polymer or polymers are selected from the group consisting of polyacrylamide (PAAM), poly(N-isopropylacrylamine) (PNIPAM), poly(vinylmethylether), poly(ethylene oxide), poly(vinylalcohol), poly(ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic Lactic Acid (PGLA) a copolymer, and a blend of two or more polymers.

3. A vaso-occlusive device as in claim 1, wherein the solid polymer is a natural polymer.

4. A vaso-occlusive device as in claim 3, wherein the natural polymer is selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin pectin, elastin, keratin, a copolymer, and a blend of polymers.

5. A vaso-occlusive device as in claim 1, wherein the pre-implantation shape comprises a shape selected from the group consisting of a strip, a rod, a sheet, a roll, a tube, a ribbon, a string, and a coil.

6. A vaso-occlusive device as in claim 1, wherein the vaso-occluding shape comprises a shape selected from the group consisting of a coil, a coiled coil, a circle, a half circle, a cone, a twisted sheet, a rod of random bends, and a helix.

7. A vaso-occlusive device as in claim 1, further comprising a bioactive agent integrated into or coating the solid material.

8. A vaso-occlusive device as in claim 7, wherein the bioactive agent comprises a bioactive agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

9. A vaso-occlusive device as in claim 8, wherein the bioactive agent comprises a tissue adhesion factor and the tissue adhesion factor is selected from the group consisting of fibrin, collagen, albumin, cyanoacrylate, fibrinogen, chitosan, and gelatin-genipin.

10. A vaso-occlusive device as in claim 1, further comprising a radio pacifier.

11. A vaso-occlusive device as in claim 10, wherein the radio pacifier comprises an agent that provides visibility of the device under X-ray or other imaging technology.

12. A vaso-occlusive device as in claim 10, wherein the radio pacifier can be identified by an imaging technique.

13. A vaso-occlusive device as in claim 1, wherein one or more of the polymers comprising the solid material comprises a biodegradable polymer.

14. A vaso-occlusive device for implantation into the vasculature of a patient to occlude abnormal blood flow comprising:
a liquid injectable polymer or combination of polymers for delivery to a site of abnormal blood flow upon which delivery the polymer polymerizes or precipitates to assume a vaso-occluding solid shape that occludes abnormal blood flow.

15. A vaso-occlusive device as in claim 14, wherein the polymer or polymers are selected from the group consisting of polyacrylamide (PAAM), poly(N-isopropylacrylamine) (PNIPAM), poly(vinylmethylether), poly(ethylene oxide), poly(vinylalcohol), poly(ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic Lactic Acid (PGLA) a copolymer, and a blend of two or more polymers.

16. A vaso-occlusive device as in claim 14, wherein the injectable polymer is a natural polymer.

17. A vaso-occlusive device as in claim 16, wherein the natural polymer is selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, pectin, elastin, keratin, a copolymer, and a blend of polymers.

18. A vaso-occlusive device as in claim 14, further comprising a bioactive agent integrated into the injectable polymer.

19. A vaso-occlusive device as in claim 18, wherein the bioactive agent comprises a bioactive agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

20. A vaso-occlusive device as in claim 19, wherein the bioactive agent comprises a tissue adhesion factor and the tissue adhesion factor is selected from the group consisting of fibrin, collagen, albumin, cyanoacrylate, fibrinogen, chitosan, and gelatin-genipin.

21. A vaso-occluding device as in claim 14, further comprising that one or more polymers comprising the resulting solid polymer are biodegradable.

22. A method of treating a patient having abnormal blood flow comprising:
implanting into the vasculature of the patient at the site of abnormal blood flow a material comprising a polymer or combination of polymers, wherein the material is either a liquid injectable that polymerizes to a solid or precipitates as a solid upon placement in the patient or is a solid material configured in a pre-implantation shape before implantation and changes to a vaso-occluding shape after implantation.

23. A method as in claim 22, wherein the material comprises a polymer or combination of polymers selected from the group consisting of polyacrylamide (PAAM), poly(N-isopropylacrylamine) (PNIPAM), poly(vinylmethylether), poly(ethylene oxide), poly(vinylalcohol), poly(ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA)), Polyacrylonitrile (PAN), Polyvinylacrylate (PVA), Polyvinylpyrrolidone (PVP), Polyglycolic Lactic Acid (PGLA), a copolymer, and a blend of two or more polymers.

24. A method as in claim 22, wherein the material is a natural polymer.

25. A method as in claim 24, wherein the natural polymer is selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, pectin, elastin, keratin, a copolymer, and a blend of polymers.

26. A method as in claim 22, wherein the material implanted in the patient comprises a bioactive agent.

27. A method as in claim 26, wherein the bioactive agent comprises a bioactive agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, and an anti-cancer factor. protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

28. A method as in claim 27, wherein the bioactive agent comprises a tissue adhesion factor and the tissue adhesion factor is selected from the group consisting of fibrin, collagen, albumin, cyanoacrylate, fibrinogen, chitosan, and gelatin-genipin.

29. A method of making a vaso-occlusive device for occluding abnormal blood flow comprised of a non-metal solid material comprising:
configuring the non-metal material into a pre-implantation shape, wherein upon implantation into a patient at a site of abnormal blood flow the material assumes a vaso-occluding shape.

30. A method as in claim 29, wherein the non-metal material is selected from the group consisting of polyacrylamide (PAAM), poly(N-isopropylacrylamine) (PNIPAM), poly(vinylmethylether), poly(ethylene oxide), poly(vinylalcohol), poly(ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic Lactic Acid (PGLA), a copolymer, and a blend of two or more polymers.

31. A method as in claim 29, wherein the non-metal material is a natural polymer.

32. A method as in claim 31, wherein the natural polymer is selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, pectin, elastin, keratin, a copolymer, and a blend of polymers.

33. A method as in claim 29, further comprising integrating into or coating the nonmetal material with a bioactive agent.

34. A method as in claim 33, wherein coating or integrating comprises a process selected from the group consisting of ion implantation, vapor deposition, plasma deposition, coating, jacketing, weaving, braiding, spraying, dipping, and spinning.

35. A method as in claim 33, wherein the bioactive agent comprises a bioactive agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

36. A method as in claim 35, wherein the bioactive agent comprises a tissue adhesion factor selected from the group consisting of fibrin, collagen, albumin, cyanoacrylate, fibrinogen, chitosan, and gelatin-genipin.

37. A method as in claim 29, wherein one or more of the polymers are biodegradable.

38. A method as in claim 29, further comprising a radio pacifier mixed into or coating the vaso-occlusive device.

39. A method of making a vaso-occlusive device for occluding abnormal blood flow in a patient comprising:
providing a liquid injectable polymer material that polymerizes to a solid or precipitates to a solid upon placement in the patient.

40. A method as in claim 39, wherein the liquid injectable polymer material is selected from the group consisting of polyacrylamide (PAAM), poly(N-isopropylacrylamine) (PNIPAM), poly(vinylmethylether), poly(ethylene oxide), poly(vinylalcohol), poly(ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic Lactic Acid (PGLA), a copolymer, and a blend of two or more polymers.

41. A method as in claim 39, wherein the material is a natural polymer.

42. A method as in claim 41, wherein the natural polymer is selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, pectin, elastin, keratin, a copolymer, and a blend of polymers.

43. A method as in claim 39, further comprising integrating into the liquid injectable material a bioactive agent

44. A method as in claim 43, wherein the bioactive agent comprises a bioactive agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

45. A method as in claim 44, wherein the bioactive agent comprises a tissue adhesion factor and the tissue adhesion factor is selected from the group consisting of fibrin, collagen, albumin, cyanoacrylate, fibrinogen, chitosan, and gelatin-genipin.

46. A method as in claim 39, wherein one or more polymers are biodegradable.

47. A method as in claim 39, farther comprising a radio pacifier mixed into or coating the vaso-occlusive device.
